(19) United States
(12) Patent Application Publication
(10) Pub. No.: US 2009/0042232 A1
(43) Pub. Date: Feb. 12, 2009

(54) MUTANT PROTEIN HAVING DIAPHORASE ACTIVITY

(75) Inventors: Yuichi Tokita, Kanagawa (JP); Yoshio Goto, Kanagawa (JP); Taiki Sugiyama, Kanagawa (JP)

Correspondence Address: BELL, BOYD & LLOYD, LLP, P. O. BOX 1135, CHICAGO, IL, 60690, US

(73) Assignee: SONY CORPORATION, Tokyo (JP)

(21) Appl. No.: 12/125,678

(22) Filed: May 22, 2008

(30) Foreign Application Priority Data
May 25, 2007 (JP) P2007-138643

(51) Int. Cl. C12Q 1/26; C12N 9/02

(52) U.S. Cl. 435/25; 435/189

(57) ABSTRACT

A mutant diaphorase capable of having lower potential energy than that of wild-type diaphorase of sequence ID No. 1 when forming a complex with a coenzyme, flavin mononucleotide.

### CROSS REFERENCES TO RELATED APPLICATIONS

The present application claims priority to Japanese Patent Application JP 2007-136843 filed in the Japanese Patent Office on May 25, 2007, the entire contents of which is incorporated herein by reference.

### BACKGROUND

The present application relates to mutant diaphorase. More specifically, the present application relates to mutant diaphorase having a predetermined level or higher of heat resistance.

Enzymes are biocatalysts for allowing many reactions for life support to smoothly proceed under mild conditions in vivo. Enzymes turn over in vivo, are produced in vivo according to need, and express their catalytic functions.

Techniques for making use of enzymes in vitro have already been used practically or studied to achieve practical use. For example, techniques for using enzymes have been developed in various technical fields, such as the production of useful materials, the production of energy-related materials, measurement or analysis, environmental conservation, and medical care. In relatively recent years, techniques, such as an enzyme cell (for example, refer to Japanese Unexamined Patent Application Publication No. 2004-71559) which is a type of fuel cell, an enzyme electrode, and an enzyme sensor (sensor for measuring a chemical substance using an enzyme reaction) have been developed.

Since the chemical bases of enzymes are proteins, enzymes have the property of being denatured by degrees of heat and pH. Therefore, enzymes have low stability in vitro as compared with other chemical catalysts such as metal catalysts. Thus, when enzymes are used in vitro, it is important to allow enzymes to more stably act in response to environmental changes and to maintain the activity.

When an enzyme is used in vitro, there are employed approaches to a method for artificially modifying the nature and function of the enzyme and a method for designing the environment of a site where the enzyme functions. In the former method, it is common that the base sequence of a gene encoding a protein is artificially modified, and the modified gene is expressed in an organism such as Escherichia coli to form an artificially mutated protein and then the mutant protein having the target function and nature is selected by screening (for example, refer to Japanese Unexamined Patent Application Publication No. 2004-298185).

In consideration of the wide availability of diaphorase in vitro, it is desirable to provide mutant diaphorase having a predetermined level or higher of heat resistance.

### SUMMARY

According to an embodiment, mutant diaphorase is capable of having lower potential energy than that of wild-type diaphorase of sequence ID No. 1 when forming a complex with a coenzyme, flavin mononucleotide.

The mutant diaphorase may be derived from, for example, thermophilic Bacillus bacteria, particularly Bacillus stearothermophilus.

According to another embodiment, a method for screening mutant diaphorase includes molecular dynamics simulation, the mutant diaphorase having lower potential energy than that of wild-type diaphorase of sequence ID No. 1 when forming a complex with the coenzyme, flavin mononucleotide.

Main technical terms will be described below.

The term “diaphorase” means an enzyme having an activity (i.e., diaphorase activity) of catalyzing an oxidation reaction of NADH or NADPH with a dye such as potassium ferricyanide, methylene blue, 2,6-dichloroindophenol, or a tetrazolium salt. The diaphorase is widely distributed in the range from microorganisms such as bacteria and yeasts to mammals. The diaphorase plays an important rule in an electron transport system in vivo. NADH or NADPH produced by a dehydrogenation reaction of a substrate caused by NAD⁺ or NADP⁺-dependent dehydrogenase is oxidized by an electron acceptor in the presence of the diaphorase, resulting in a reduced form of the electron acceptor.

Flavin mononucleotide (referred to as “FMN” hereinafter) acts as a coenzyme of an oxidoreductase called a flavin enzyme in vivo. The term “coenzyme” means a low-molecular-weight organic compound functioning to give and receive a chemical group in an enzyme reaction. The coenzyme and an apoenzyme (enzyme protein portion lacking a coenzyme) do not function independently as a chemical reaction catalyst, but both are bonded together to form a holoenzyme which functions as an enzyme. Examples of the flavin enzyme having FMN as a coenzyme include L-amino acid dehydrogenase and glycolate oxydase.

The term “mutant protein” means a protein expressed from a gene produced by artificially modifying the base sequence of DNA which encodes an amino acid sequence constituting a protein. More specifically, the term means a protein having an amino acid sequence in which at least one amino acid residue in a wild-type amino acid sequence is lost, substituted, or added.

The molecular dynamics simulation is adopted for reproducing motions of each of atoms which constitute a system (gas, liquid, or solid) on a computer. The Newton's laws of motion are resolved for each of atoms and molecules to permit a simulation of time development of a state of a target system. As a result, information on the higher-order structure of a protein is obtained with high resolution which is not obtained by experiments. The specific simulation method used will be described in detail in Example 4. However, the simulation method is not particularly limited this, and general program and force field may be used

Molecules tend to return to an energetically stable state (equilibrium state) and thus are energetically unstable and have excessive energy in a state shifting from a natural bond state. This excessive energy is referred to as “potential energy”. The potential energy is represented as a function (potential function) which is expressed in classical mechanics. The potential function used in the molecular dynamics simulation varies depending on the program and force field used. The potential function used is described in detail in Example 4.

The mutant diaphorase according to an embodiment has a predetermined level or higher of heat resistance.

Additional features and advantages are described herein, and will be apparent from the following Detailed Description and the figures.

### BRIEF DESCRIPTION OF THE FIGURES

FIG. 1 is a schematic diagram showing the flow of an experiment according to Example 2 (library preparation by random mutation and screening);

FIG. 2 is part of a photograph (an alternative to a drawing) of a well plate when a total of about 8,000 colonies are screened;

FIG. 3 is part of a photograph of a well plate resulting from a double check experiment;

FIG. 4 shows tables, as an alternative to a drawing, summarizing factors that reflect ease of binding of substrates to active sites of wild-type diaphorase and G122D mutant diaphorase;

FIG. 5 is a photograph as an alternative to a drawing showing the structure of wild-type diaphorase obtained by X-ray crystallography; and

FIG. 6 is a photograph as an alternative to a drawing showing a portion of the conformation of wild-type diaphorase obtained by simulation.

### DETAILED DESCRIPTION

It has been found that diaphorase is present as a dimer in which two molecules of FMN are bonded at an interface between sub-units to form a complex. Therefore, the inventors studied various types of mutant diaphorase with respect to potential energy and heat resistance when forming a complex with FMN.

In other words, it has been found that the heat resistance of diaphorase depends on the potential energy, and the heat resistance of diaphorase improves as the potential energy decreases.

Hereinafter, description will be made with reference to experiment data.

### EXAMPLES

### Example 1

Cloning, expression, and purification of diaphorase derived from Bacillus stearothermophilus

(1-1) Isolation and purification of genomic DNA from Bacillus stearothermophilus

Bacillus stearothermophilus was purchased from Japan Collection of Microorganisms (JCM), Riken (JCM No. 2501, NCBI accession number of diaphorase gene: AFI 12858). A lyophilizate of Bacillus stearothermophilus was cultured on agar medium A overnight at 55° C.

The resulting colony was similarly cultured on fresh agar medium A to form a pure colony. A portion of the colony was picked up, cultured in liquid medium A overnight at 55° C., and centrifuged to collect bacteria. Genomic DNA was isolated with Wizard Genomic DNA Purification Kit (Promega Corporation) (details of the process are described in an instruction manual attached to a product). The composition of the medium A was as shown in Table 1 (pH 7.0 to 7.2 in 1 L).

TABLE 1

| | |
|---|---|
| Meat Extract (Merck) | 10 g |
| Bacto Peptone (DIFCO) | 10 g |
| NaCl (Wako) | 5 g |
| Agar, guranulated (DIFCO) | 20 g (in the case of an agar medium) |

### (1-2) Cloning of Diaphorase Gene

A diaphorase gene was amplified by PCR from the genomic DNA obtained in (1-1). Pfu DNA polymerase (Stratagene) was used as a DNA polymerase. A primer having the sequences shown in Table 2 was used. Underlined portions represent Nde I sequence (sense_DI) and BamH I sequence (antisense_DI).

TABLE 2

| | | |
|---|---|---|
| sense_DI | ggaat tccat atgat | Sequence ID No. 57 |
| | gacaa acgta ttgta | |
| | cat | |
| antisense_DI | cggga tcctt aaaac | Sequence ID No. 58 |
| | gtgtg cgcca agt | |

Next, the diaphorase gene which was a PCR product was purified with PCR Cleanup Kit (Qiagen) and confirmed by agarose electrophoresis. Further, the base sequence was confirmed with a DNA sequencer.

### (1-3) Introduction of Diaphorase Gene into Vector

Amplified fragments of the diaphorase gene were treated with BamH I and Nde I and purified with PCR Cleanup Kit (Qiagen). In addition, vector pET12a (Novagen) was similarly treated with BamH I and Nde I and purified. The resulting two types of fragments were ligated with T4 ligase, and XL1-blue electrocompetent cells (Stratagene) were transformed with the products and cultured in an LB-amp medium to increase production.

The resulting plasmid was treated with Bss I, and insertion of the diaphorase gene was confirmed by agarose electrophoresis. The base sequence was analyzed. The results showed a slight difference between the resulting base sequence and a base sequence in database (NCBI). This was possibly due to the fact that because the strain purchased from Japan Collection of Microorganisms, Riken, is slightly different from a strain described in the database, the gene sequence of the cloned DI was inconsistent with the gene sequence of DI in the database. In the genotype (base sequence), there were inconsistencies at 11 positions. Among these, in the phenotype (amino acid sequence), there were inconsistencies at two residues (see Table 3).

TABLE 3

| Amino acid residue No. | Acquired gene | Database |
|---|---|---|
| 28 | Glutamic acid | Aspartic acid |
| 61 | Aspartic acid | Glycine |

Therefore, a gene was formed with Quick Change Site-Directed Mutagenesis Kit (Stratagene) so that the amino acid residues at the two positions were modified to be identical to those in the database. The resultant gene was named “pET12a-BsDI.”

### (1-4) Transformation of Escherichia coli

The gene pET12a-BsDI was introduced and transformed into E. coli BL21 (DE3) (Novagen) by a heat shock method. After preculture in SOC at 37° C. for 1 hour, the resultant transformant was developed on an LB-amp agar medium, and part of the colonies was cultured in a liquid medium. The expression of diaphorase was confirmed by SDS-PAGE.

### (1-5) Cryopreserved Sample of Transformant

First, 3 mL of a culture solution of the transformant formed in (1-4) was centrifuged. Then, E. coli pellets were dispersed in a 2xYT medium, and the resultant dispersion was stored at −80° C.

### (1-6) Mass Culture and Purification of Protein

The frozen sample of BL21(DE3)/pET12a-BsDI was developed on an LB-amp agar medium, and the colonies were picked up and precultured in 100 mL of an LB-amp medium until OD600 reached about 1. The preculture was developed on 18 L of an LB-amp medium and cultured at 37° C. by shaking until OD600 was saturated at about 2. The culture was centrifuged at 5 kG to recover bacteria (wet yield, 20 g). The bacteria pellets were frozen at −80° C., melted, and then sonicated at 0° C. in 200 mL of a solution containing 50 mM Tris-HCl at pH 7.8, 1 mM EDTA, 1 mM DTT, and 1 mM PMSF to cause bacteriolysis. Then, a solution fraction was recovered by centrifugation (9.5 kG).

Next, purification was performed by an ammonium sulfate precipitation method. First, powdery ammonium sulfate was gradually added under stirring to prepare a 35% saturated solution. The resultant saturated solution was allowed to stand overnight, and precipitates were removed by centrifugation (9.5 kG). The residue was desalted with a dialysis membrane tube (final solution: 5 mM Tris-HCl, pH 7.8). Then, 50 mL of a sample concentrated by ultrafiltration was passed through an anion-exchange column (Sepharose Q FastFlow, Amersham Bioscience) to recover a diaphorase-containing fraction. The resultant fraction was concentrated by ultrafiltration (amount of the solution: 20 mL, Centriplus centrifugal filter unit YM-30, Millipore). Then, the resultant sample was passed through a gel filtration column (Sephacryl S-200, Amersham Bioscience) to collect a diaphorase-containing fraction.

### Example 2

Preparation of mutant library by random mutation of diaphorase derived from Bacillus stearothermophilus and screening of heat-resistant mutant.

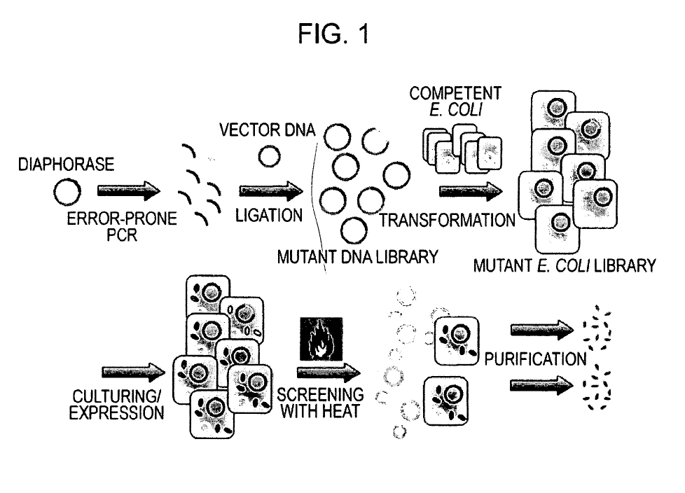

FIG. 1 shows the flow of an experiment conducted in Example 2. A gene library of diaphorase mutants was prepared by error-prone PCR. The gene was introduced into vector DNA and expressed in Escherichia coli. The resultant library was subjected to screening for heat resistance to extract a target heat-resistant diaphorase mutant.

### (2-1) Error-Prone PCR with GeneMorph (Registered Trade Name)

The error-prone PCR method is a method for randomly mutating replicate DMA fragments using misreading of a base sequence by DNA polymerase in replication reaction of DNA fragments by PCR. A variety of methods has been reported, but GeneMorph (registered trade name, Stratagene) was selected from commercially available methods. The pE12a-BsDI integrated with the diaphorase gene of Bacillus stearothermophilus was used as a template DNA. The primer used for cloning the gene was also used.

Table 4 shows the sequences of the primer. The primer has the sequence of Nde I at the 5′-terminus of sense_DI and the sequence of BamH I at the 5′-terminus of antisense_DI (underlined portions). Thus, error-prone PCR products may be inserted into a multicloning site of pFT12a by treatment with these restriction enzymes (similar to cloning of wild-type diaphorase).

TABLE 4

| | | |
|---|---|---|
| sense_DI | ggaat tccat atgat | Sequence ID No. 59 |
| | gacaa acgta ttgta | |
| | cat | |
| antisense_DI | cggga tcctt aaaac | Sequence ID No. 60 |
| | gtgtg cgcca agt | |

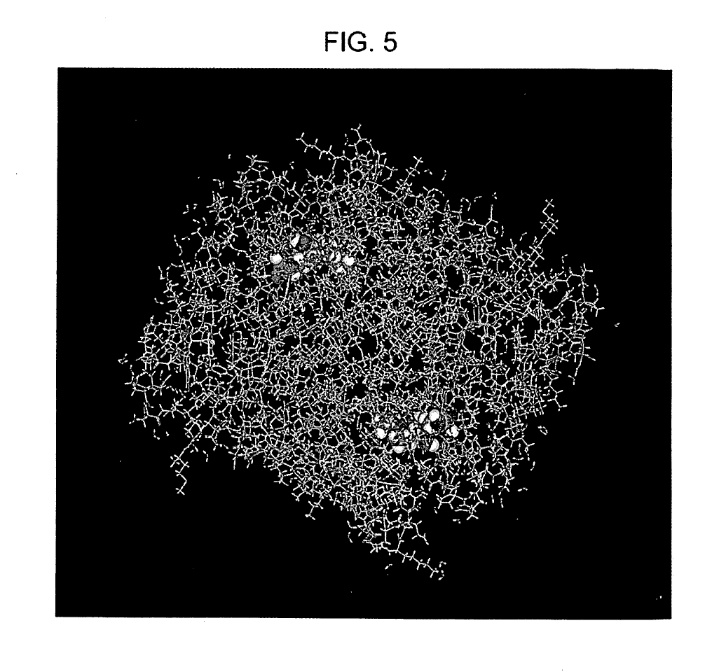

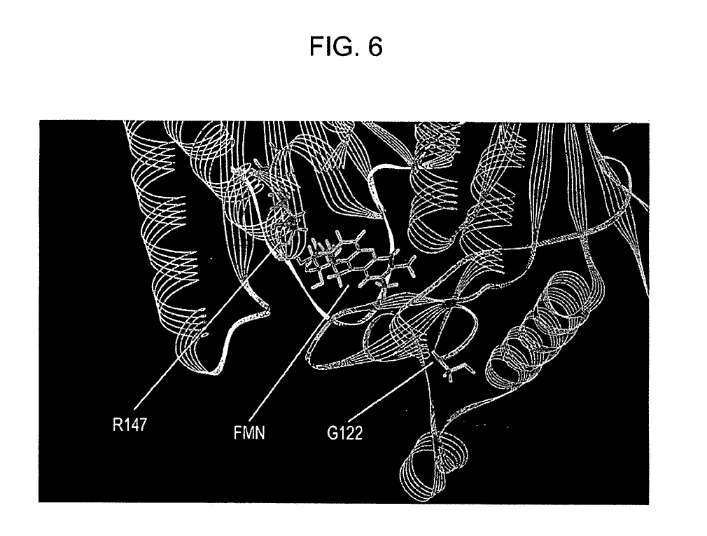

PCR was performed according to the manual of GeneMorph (registered trade name) on the basis of preparation of a reaction solution shown in FIG. 5 and a thermal cycle shown in FIG. 6.

TABLE 5
<Preparation of reaction solution>

| | |
|---|---|
| 41.5 μL | water |
| 5.0 μL | 10X Mutazyme reaction buffer |
| 1.0 μL | 40 mM dNTP mix (200 μM each final) |
| 0.5 μL | primer mix (250 ng/μL of each primer) |
| 1.0 μL | Mutazyme DNA polymerase (2.5 U/μL) |
| 1.0 μL | template (10 pg/μL-10 ng/μL) |
| 50.0 μL | in total |

TABLE 6
<Thermal cycle>

| | Number of | | |
|---|---|---|---|
| Segment | cycle | Temperature | duration |
| 1 | 1 | 96° C. | 30 sec |
| 2 | 30 | 96° C. | 30 sec |
| | | 53° C.*¹ | 30 sec |
| | | 72° C. | 1 min*² |
| 3 | 1 | 72° C. | 10 min |

*¹Primer Tm −5° C.
*²1 min for ≦1 kb target

### (2-2) Introduction of Diaphorase Gene into Vector

The total amount of the error-prone PCR products excluding the amount used for agarose gel electrophoresis was used for treatment with the restriction enzymes Nde I and BamH I. After reaction at 37° C. for 2 hours, the reaction product was purified by Qiaquick PCR purification kit (Qiagen). On the other hand, the vector pET12a was treated with the restriction enzymes Nde I and BamH I in the same manner as for the PCR products (at 37° C. for 2 hours).

The reaction products of the treatment with the restriction enzymes were separated by low-melting-point agarose gel electrophoresis. The corresponding open-ring vector DNA was purified with Qiaquick Gel Extraction Kit (Qiagen). Next, the products of treatment of the purified vector with the restriction enzymes were dephosphorylated at the 5′-terminus by treatment with alkali phosphatase. The reaction products were purified with Qiaquick PCR purification Kit (Qiagen). The resulting error-prone PCR products, i.e., the diaphorase mutant gene library, were ligated into the vector which was treated with the restriction enzymes and dephosphorylated. The ligation reaction was performed with Ligation Kit Mighty Mix (Takara Bio Inc.). The reaction product was purified by an ethanol precipitation method.

### (2-3) Preparation of Competent Cell and Transformation

Electrocompetent cells (competency, about 10⁸/ng) of BL21 (DE3) self-prepared were used as competent cells. Next, 40 μL of a competent cell frozen sample was melted on ice, and 0.5 μL of a DNA sample at a concentration of about 1 μg/μL was mixed thereto. The total of the resultant mixture was set in an electroporation cuvette with a gap of 0.1 cm, and transformation was performed by applying a voltage of 1800 kV. Then, 960 μL of a SOC medium was added to the mixture, followed by preculture with shaking at 37° C. for 1 hour. The culture solution was inoculated into 5 to 10 μL of an LB-amp agar medium and then incubated at 37° C. overnight.

### (2-4) Screening Method

Each of the single colonies on the agar medium obtained in (2-3) was inoculated into an LB-amp liquid medium (150 μL) of a 96-well plate using a toothpick. Two wells were used for a strain of Escherichia coli that produces a wild type. The top of the well plate was sealed with a gas-permeable adhesive sheet (ABgene) and further covered with an accompanying lid, followed by shaking culture at 37° C. overnight (about 14 hours). Then, 25 μL of each culture solution was sufficiently mixed with the same amount of a 0.2N NaOH aqueous solution (previously dispensed) on a new well plate by pipetting. The plate was covered with a lid and then incubated with an incubator at 37° C. for 15 minutes to cause bacteriolysis.

Next, 100 μL of 0.1 M K-pi at pH 6.8 was added at room temperature to neutralize the mixture. One of the two wild-type samples was separated as an unheated control sample, charged in a microtube, and stored at room temperature. Then, the plate was sealed with a commercial OPP tape, heat-treated with an incubator at 80° C. for 75 minutes, and then cooled by allowing to stand at room temperature. Then, the separated wild-type sample was returned to the plate. Then, 10 μL of a 20 mM anthraquinone sulfonic acid (AQS) 20 DMSO solution and 50 μL of a 80 mM NADH aqueous solution which was prepared immediately before use were added to each sample in order. The plate was sealed with an OPP tape, and the resultant mixture was stirred with vortex mixer for 5 seconds. Color development was recorded with a camera, and samples with strong coloration by reduction of AQS as compared with the wild-type sample were selected as candidates.

### (2-5) Preservation of Sample

In samples passing through screening, part of the culture solution remaining on the 96-well plate was inoculated into 4.5 mL of a LB medium, and cultured overnight. The plasmid was purified and stored in a refrigerator. In addition, the culture solution was separately inoculated into 4 mL of an LB medium, cultured until OD600 reached about 0.4, and then centrifuged to collect bacteria. The resulting bacteria were suspended in 2 mL of a 2xYT medium, frozen with liquid nitrogen, and stored at −80° C.

### (2-6) Abundant Expression and Purification of Diaphorase Mutant

Abundant expression and purification of a diaphorase mutant were performed by a method described above. However, in the abundant expression, E. coli was cultured in 1 L of LB-amp, and the volume and the like in each step of subsequent purification were adjusted according to the culture scale.

### (2-7) Activity Evaluation Test

The activity of diaphorase was evaluated by the rate (the number of moles of the product produced by a reaction catalyzed by 1 mol of enzyme per unit time (unit: sec⁻¹)) of a catalytic reaction in which nicotinamide dinucleotide oxidized form (NAD⁺) and 2-amino-1,4-dihydroxynaphthene were produced by reduction reaction of 2-amino-1,4-naphthoquinone (ANQ) with nicotinamide dinucleotide reduced form (NADH). The evaluation was performed under the following conditions: A reaction solution contained 100 mM K-pi at pH 8.0 ([ANQ]=0.3 mM, [NADH]=40 mM, [diaphorase]=48 mM). Deoxygenation was sufficiently performed by argon bubbling before measurement, and the reaction was performed at 25° C. in an argon atmosphere. The reaction was initiated by adding diaphorase, and the progress of the reaction was monitored by measuring decreases in absorbance (molar absorption coefficient 680 M⁻¹cm⁻¹) of ANQ at 520 nm to calculate the reaction rate.

### (2-8) Heat Resistance Test

A solution of purified diaphorase mutant sample in 50 mM Tris-HCl at pH 7.8 and a 300 mM NaCl solution was concentrated by ultrafiltration and buffer-exchanged to prepare a 0.1 M K-pi solution at pH 8.0. The resultant solution was appropriately diluted so that the absorbance of diaphorase at 460 nm was 0.1 to prepare a solution at an enzyme concentration of 8.3 μM. The resultant solution was incubated with an aluminum block heater or the like at 80° C. for 10 minutes, immediately cooled on ice, and then sufficiently cooled, followed by the measurement of activity. Also, a control experiment was performed using a sample not incubated. With respect to the ratio of residual enzyme activity, the enzyme activity was measured under the same conditions before and after heating, and the ratio of the activity value after heating to the value before heating was represented by percent.

### (2-9) Results

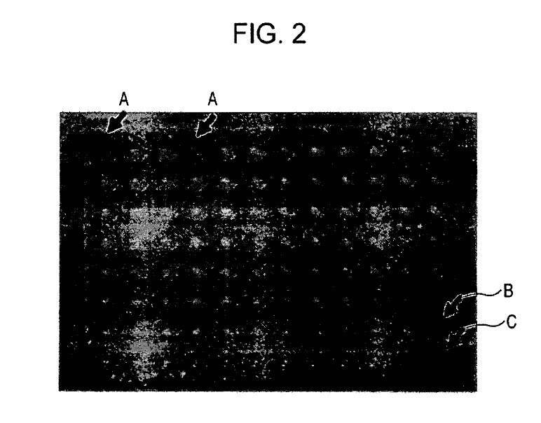

A total of about 8,000 colonies was screened according to the above-described method. FIG. 2 shows part of a photograph of a well plate used in the example. FIG. 2 shows an example of detection of diaphorase maintaining activity during screening. Arrows A indicate samples as heat-resistant mutant candidates detected on the plate, arrow B indicates a wild-type sample as a control, and arrow C indicates a wild-type sample not heated.

In consideration of possible errors, such as error between the plates, differences in diaphorase expression between strains, and the like, the selected samples were screened again at a time. Namely, cryopreserved E. coli samples were streak-cultured on a LB agar medium, and the resultant colonies were inoculated into a 96-well plate and heated. However, in order to minimize errors, 8 colonies per sample were screened.

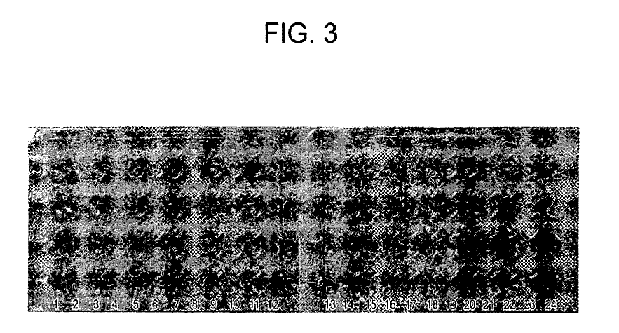

FIG. 3 shows part of a photograph of the results of the double check test. In FIG. 3, the same mutant sample was disposed in a column. As a control, the wild-type samples after heating were disposed in column No. 11 and 4 wells on the upper side of column No. 24, and the wild-type samples not heated were disposed in column No. 12 and 4 wells on the lower side of column No. 24. In the example of the photograph shown in FIG. 3, column Nos. 7, 14, 17, 19, 20, and 21 were positive and thus selected as heat-resistant mutant candidates.

Tables 7 to 12 show the results of the heat resistance test on the heat-resistant diaphorase mutant candidates obtained as described above.

TABLE 7

| Sequence | | Activity | Ratio of residual enzyme |
|---|---|---|---|
| ID No. | Type of mutant | (S⁻¹) | activity (%) |
| 1 | WT (wild type, control) | 168 | 23 |
| 2 | K139N/A187E | 367 | 53 |
| 3 | F105L | 246 | 48 |
| 4 | G122D | 362 | 28 |
| 5 | G131E | 250 | 28 |
| 6 | A146G | 263 | 9 |
| 7 | R147H | 315 | 4 |
| 8 | H34Q | 228 | 8 |
| 9 | F105H | 283 | 33 |
| 10 | A113E | 143 | 46 |

TABLE 8

| Sequence ID | | Activity | Ratio of residual |
|---|---|---|---|
| No. | Type of mutant | (S⁻¹) | enzyme activity (%) |
| 11 | K123E | 155 | 34 |
| 12 | K139N | 263 | 25 |
| 13 | R147S | 226 | 17 |
| 14 | G149D | 168 | 22 |
| 15 | G154D | 247 | 24 |
| 16 | A156E | 318 | 27 |
| 17 | M159T | 196 | 28 |
| 18 | A187E | 407 | 52 |
| 19 | A187T | 328 | 17 |
| 20 | A187V | 214 | 33 |

TABLE 9

| Sequence | | Activity | Ratio of residual |
|---|---|---|---|
| ID No. | Type of mutant | (S⁻¹) | enzyme activity (%) |
| 21 | R64H/A146T | 169 | 20 |
| 22 | E85D/R147H | 321 | 37 |
| 23 | F105L/A187E | 241 | 46 |
| 24 | A113E/K126N | 211 | 31 |
| 25 | Y151H/A187E | 284 | 20 |
| 26 | G122D/A187E | 356 | 61 |
| 27 | G149D/A187E | 215 | 53 |
| 28 | G149S/A187E/L207W | 212 | 38 |
| 29 | F105L/A187E/L207W | 524 | 15 |
| 30 | G66R/F105L/A187E/K192R | 428 | 24 |

TABLE 10

| Sequence | | Activity | Ratio of residual |
|---|---|---|---|
| ID No. | Type of mutant | (S⁻¹) | enzyme activity (%) |
| 31 | A146G/L207W | 213 | 15 |
| 32 | F105L/A187E/Q171P | 283 | 68 |
| 33 | A78E/F105L/A187E | 284 | 68 |
| 34 | F105L/K149N/V168L/A187E | 270 | 55 |
| 35 | G154D/G180R | 297 | 33 |
| 36 | F107I | 283 | 53 |
| 37 | G185R | 446 | 58 |
| 38 | Y151H/G185R | 315 | 15 |
| 39 | G122D/G185R | 387 | 63 |
| 40 | G149D/G185R | 264 | 54 |

TABLE 11

| Sequence | | Activity | Ratio of residual |
|---|---|---|---|
| ID No. | Type of mutant | (S⁻¹) | enzyme activity (%) |
| 41 | G149D/G185R/A208V | 223 | 26 |
| 42 | F107I/G185R | 305 | 48 |
| 43 | F107I/G185R/A208V | 545 | 73 |
| 44 | F107I/G185R/Q171P | 410 | 21 |
| 45 | V80D/F107I/G185R | 437 | 72 |
| 46 | F107I/K139N/V168L/G185R | 283 | 47 |
| 47 | F150V | 380 | 58 |
| 48 | A193E | 497 | 63 |
| 49 | F150V/A193E | 412 | 51 |
| 50 | Y151H/A193E | 358 | 18 |

TABLE 12

| Sequence | | Activity | Ratio of residual |
|---|---|---|---|
| ID No. | Type of mutant | (S⁻¹) | enzyme activity (%) |
| 51 | G122D/A193E | 418 | 68 |
| 52 | G149D/A193E/A208V | 275 | 30 |
| 53 | F150V/A193E/A208V | 572 | 78 |
| 54 | F150V/A193E/Q171P | 458 | 43 |
| 55 | V80D/F150V/A193E | 512 | 82 |
| 56 | K139N/F150V/V168L/A193E | 294 | 37 |

As a result, for example, in mutant diaphorase having the amino acid sequences of sequence ID Nos. 2 to 7, 9, 12, 15, 16, 18, 19, 22, 25, 26, 29, 30, 32 to 40, and 42 to 56, enzyme activity (reaction rate) was significantly improved as compared with the wild type (WT). In addition, for example, in mutant diaphorase having the amino acid sequences of sequence ID Nos. 2, 3, 18, 23, 26, 27, 32 to 34, 36, 37, 39, 40, 42, 43, 45 to 49, 51, and 53 to 55, the ratio of residual enzyme activity after heat treatment was particularly excellent as compared with the wild type (WT).

Furthermore, in the experimental system, a target diaphorase mutant having improved enzyme activity was successfully obtained. Therefore, it was confirmed that the method for preparing a mutant library by random mutation using error-prone PCR and the method for screening by heat treatment are effective.

### Example 3

Detailed investigation of heat-resistant diaphorase mutant

Among the mutants obtained by the above-described research, G122D having improved enzyme activity as compared with the wild type was investigated on the basis of enzyme kinetics.

First, the ANQ concentration under the condition of 40 mM NADH was changed to various values to plot enzyme reaction rates. In addition, the NADH concentration under the condition of 2.2 mM ANQ was changed to various values to plot enzyme reaction rates. The results well coincided with the Michaelis-Menten equation. Further, kcat, KM(NADH), and KM(ANQ) were determined on the basis of the equation.

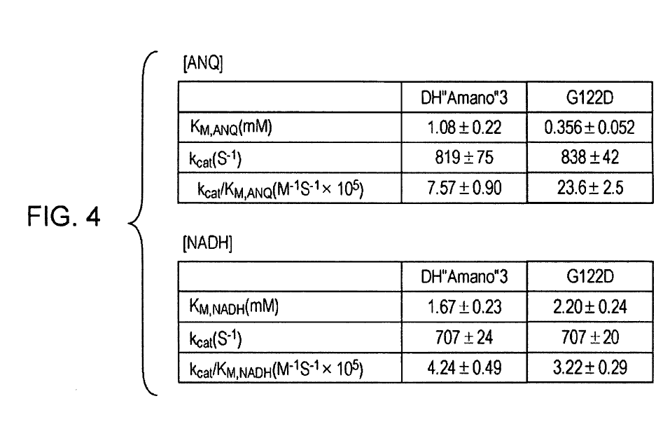

For comparison, kcat, KM(NADH), and KM(ANQ) of wild-type diaphorase (DI(DH“Amano”3) are also shown. Diaphorase takes a so-called ping-pong type reaction system. The term “kcal” represents a turnover number per unit time of a catalytic reaction. The terms “KM(NADH)” and “KM(ANQ)” refer to Michaelis constants for substrates and are factors that reflect the ease of bonding of the substrates to active sites of the enzymes. The results are summarized in attached FIG. 4 (an alternative to a drawing).

These results show that the mediator ANQ bonding site of the mutant has the property of ease of bonding as compared with the wild type (refer to the ANQ-related table of FIG. 4). On the other hand, with respect to the NADH bonding site, particularly no change from the wild type was observed or reduction was rather observed (refer to the NADH-related table of FIG. 4). Therefore, it may be predicted that the higher catalytic ability of the mutant (mutant diaphorase) is due to the acquisition of affinity for the substrate ANQ.

This result means that the mutant does not exhibit higher activity at higher concentrations but exhibits an advantage at a low concentration. For example, this may lead to the advantage that the concentration of mediator ANQ in an enzyme cell may be suppressed.

### Example 4

Study by molecular dynamics simulation

In this example, the conformations of wild-type diaphorase and mutant diaphorase (G122D) (sequence ID No. 4), and R147H (sequence ID NO. 7) were estimated by molecular dynamics simulation. The potential energy of each of the wild type and mutant was calculated, and a relation between potential energy and heat resistance was examined by comparison.

Outlines of a calculation method and calculation model for molecular dynamics simulation will be described below.

In this simulation, a commercial protein modeling software, Discovery Studio Modeling (referred to as “DS Modeling” hereinafter), was used. “DS Modeling 1.6” was used for calculation and analysis using a force field.

As an initial structure, a structure (refer to FIG. 5) obtained by X-ray crystallography was used.

Next, the initial structure was optimized by assigning CHARMm (Chemistry at Harvard Macromolecular Mechanics) force field to each atom and molecular mechanical calculation. The CHARMm is described in detail in the following reference document; CHARMM: A program for Macromolecular Energy Minimization and Dynamics Calculations (Books et al. 1983, Journal of Computational Chemistry, 4: 187-217).

The structure was first optimized by 1,000 steps of (1 step is 1 femtosecond) calculation by a steepest decent method and then 5,000 steps of calculation by an adapted basis Newton-Raphson method.

Next, in order to consider thermodynamic conditions, the set condition was changed from 50 K to 300 K through 2,000 steps, and the structure at 300 K was calculated.

Next, the number n of particles, volume V, and temperature T were set to constant values (NVT ensemble), and equilibrium calculation at 300 K was performed through 1,000 steps (1 step is 1 femtosecond).

Next, MD (Molecular Dynamics) calculation was performed by the NVT ensemble through 1,000,000 steps, and the motion of each atom was tracked to perform energy analysis.

Here, the potential energy defined by the CHARMm force field is described. Details thereof are described in the above-described reference document. In the CHARMm force field, the potential energy (Eₜₒₜₐₗ) is represented by the equation (1) below. Namely, the potential energy (Eₜₒₜₐₗ) is represented as a total of functions such as bond stretching potential term (E_{bond}), bond bending potential term (E_{angle}), torsion angle potential term (Eₜₒᵣₛᵢₒₙ), out-of-plane deformation potential term (Eᵢₘₚᵣ), and nonbonding potential term (E_{nonbond}).

Eₜₒₜₐₗ=E_{bond}+E_{angle}+Eₜₒᵣₛᵢₒₙ+Eᵢₘₚᵣ+E_{nonbond} Equation (1)

Further, the potential functions will be described by the following respective equations (2) to (10):

E_{bond}=Σk_{b}(r−r₀)² Equation (2)

E_{angle}=E_{θ}+E_{UB} Equation (3)

E_{θ}=Σk_{θ}(θ−θ₀)² Equation (4)

E_{UB}=ΣK_{UB}(S−S₀)² Equation (5)

Eₜₒᵣₜᵢₒₙ=Σ_{Φ}(1+cos(nΦ−δ)) Equation (6)

Eᵢₘₚᵣ=Σk_{ω}(ω−ω₀)² Equation (7)

E_{nonbound}=E_{elec}+E_{vdw} Equation (8)

$$\begin{matrix}{E}_{elec} = {\sum }_{excl ( i , j ) = 1} \frac{{q}_{i} {q}_{j}}{ɛ {r}_{ij}} & Equation ( 9 ) \\ {E}_{vdw} = \sum ɛ [ {( \frac{{R}_{{min}_{ij}}}{{r}_{ij}} )}^{12} - {( \frac{{R}_{{min}_{ij}}}{{r}_{ij}} )}^{6} ] & Equation ( 10 )\end{matrix}$$

The parameters in the functions are described in brief below.
(1) E_{bond} (Bond potential energy)
k_{b}: force constant, r: bond distance (r₀: equilibrium value)
(2) E_{θ} (Bond angle potential energy)
k_{θ}: force constant, θ: bond angle (θ₀: equilibrium value)
(3) E_{UB} (Urey-Bradley angle potential energy)
K_{UB}: force constant, S: nonbonded interatomic distance (S₀: equilibrium value)
(4) Eₜₒᵣₛᵢₒₙ (Dihedral angle potential energy)
K_{Φ}: torsion barrier, n: periodicity, Φ: torsion angle, δ: phase
(5) Eᵢₘₚᵣ (Improper torsion angle potential energy)
k_{ω}: force constant, ω: dihedral angle (ω₀: equilibrium value)
(6) E_{elec} (Electrostatic energy)
q: charge, ε: dielectric constant, rᵢⱼ: distance between atoms i and j
(7) E_{vdw} (van der Waals energy)
ε: depth of potential well of atom pair, Rminᵢⱼ: distance between atoms i and j with the most stable energy, rᵢⱼ: distance between atoms i and j

FIG. 5 is a photograph, as an alternative to a drawing, showing the structure of the wild-type diaphorase obtained by X-ray crystallography. The results of analysis indicate that diaphorase is present as a dimer. In this figure, C-C bonds of sub-units are respectively shown in green and gray.

The analysis further indicates that two molecules of FMN are bonded at an interface between the sub-units of the dimer to form a complex composed of two molecules of diaphorase and two molecules of FMN. In the figure, FMN is shown by space-fill.

FIG. 6 is a photograph, as an alternative to a drawing, showing a portion of the conformation of the wild-type diaphorase obtained by simulation. This conformation is a final conformation after MD calculation. In FIG. 6, “FMN” shows the position of FMN, and “R147” and “G122” each show the position of an amino acid residue substituted in the mutant protein.

With respect to the wild type and each mutant diaphorase, the results of MD calculation of potential energy using the above-described force field are shown in Table 13.

TABLE 13

| | | Ratio of residual enzyme |
|---|---|---|
| Type of mutant | Potential energy (kcal/mol) | activity (%) |
| R147H | −2591.7 | 4 |
| WT (wild type) | −2688.9 | 23 |
| G122D | −2717.6 | 28 |

The potential energy (−2717.6 kcal/mol) of the G122D mutant diaphorase is lower than the potential energy (−2688.9 kcal/mol) of the wild-type diaphorase. In contrast, the potential energy (−2591.7 kcal/mol) of the R147H mutant diaphorase is higher than that of the wild-type diaphorase.

The ratio of residual enzyme activity of each of the wild type and mutant diaphorase determined by the heat resistance test in Example 2 is shown in the right column in Table 13.

The heat resistance (ratio of residual enzyme activity, 28%) of the G122D mutant diaphorase is improved as compared with that (ratio of residual enzyme activity, 23%) of the wild-type diaphorase. In contrast, the heat resistance (ratio of residual enzyme activity, 4%) of the R147H mutant diaphorase is significantly decreased.

These results significantly suggest that the enzyme activity of diaphorase depends on the potential energy, and the heat resistance of diaphorase improves as the potential energy decreases.

These findings indicate that mutant diaphorase having lower potential energy exhibits excellent heat resistance as compared with wild-type diaphorase. This shows that in mutant proteins in which various amino acid mutations are introduced, the potential energy is calculated by molecular dynamics simulation, and mutants having lower potential energy that of wild-type diaphorase are screened to obtain mutant diaphorase having improved heat resistance.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A mutant diaphorase capable of having lower potential energy than that of wild-type diaphorase of SEQ ID No: 1 when forming a complex with a coenzyme, flavin mononucleotide.

2. The mutant diaphorase according to claim 1, wherein the potential energy is lower than −2688 kcal/mol.

3. The mutant diaphorase according to claim 1, wherein the mutant diaphorase is derived from thermophilic Bacillus bacteria.

4. The mutant diaphorase according to claim 3, wherein the mutant diaphorase is derived from Bacillus stearothermophilus.

5. A method for screening mutant diaphorase using molecular dynamics simulation, the mutant diaphorase being capable of having lower potential energy than that of wild-type diaphorase of SEQ ID No: 1 when forming a complex with a coenzyme, flavin mononucleotide.


SEQUENCE LISTING (60 sequences)

SEQ ID NO: 1 - protein, 211 aa, Bacillus stearothermophilus
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 2 - protein, 211 aa, Artificial Sequence
  K139N/A187E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDNKALHIQARGGFYSEGPAAEMEMGHRYLSVIMQFFGVPSFEGLFVEGHEAVPEKAEEIKANAIARAKDLAHTF

SEQ ID NO: 3 - protein, 211 aa, Artificial Sequence
  F105L
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNLSFPPVLKAYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 4 - protein, 211 aa, Artificial Sequence
  G122D
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVADKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 5 - protein, 211 aa, Artificial Sequence
  G131E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQEPVGLLTDKKALHIQARGGFYSEGPAAEMEMGHRYLSVIMQFFGVPSFEGLFVEGHAAVPEKAEEIKANAIARAKDLAHTF

SEQ ID NO: 6 - protein, 211 aa, Artificial Sequence
  A146G
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQGRGGFYSEGPAAEMEMGHRYLSVIMQFFGVPSFEGLFVEGHAAVPEKAEEIKANAIARAKDLAHTF

SEQ ID NO: 7 - protein, 211 aa, Artificial Sequence
  R 147H
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQAHGGFYSEGPAAEMEMGHRYLSVIMQFFGVPSFEGLFVEGHAAVPEKAEEIKANAIARAKDLAHTF

SEQ ID NO: 8 - protein, 211 aa, Artificial Sequence
  H34Q
MTNVLYITAHPHDDTQSYSMAVGKAFIDTYKQVQPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 9 - protein, 211 aa, Artificial Sequence
  F105H
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNHSFPPVLKAYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 10 - protein, 211 aa, Artificial Sequence
  A 113E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKEYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 11 - protein, 211 aa, Artificial Sequence
  K123E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGETFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 12 - protein, 211 aa, Artificial Sequence
  K139N
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDNKALHIQARGGFYSEGPAAEMEMGHRYLSVIMQFFGVPSFEGLFVEGHAAVPEKAEEIKANAIARAKDLAHTF

SEQ ID NO: 13 - protein, 211 aa, Artificial Sequence
  R147S
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQASGGFYSEGPAAEMEMGHRYLSVIMQFFGVPSFEGLFVEGHAAVPEKAEEIKANAIARAKDLAHTF

SEQ ID NO: 14 - protein, 211 aa, Artificial Sequence
  G 149D
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGDFYSEGPAAEMEMGHRYLSVIMQFFGVPSFEGLFVEGHAAVPEKAEEIKANAIARAKDLAHTF

SEQ ID NO: 15 - protein, 211 aa, Artificial Sequence
  G154D
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEDPAAEMEMGHRYLSVIMQFFGVPSFEGLFVEGHAAVPEKAEEIKANAIARAKDLAHTF

SEQ ID NO: 16 - protein, 211 aa, Artificial Sequence
  A156E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPEAEMEMGHRYLSVIMQFFGVPSFEGLFVEGHAAVPEKAEEIKANAIARAKDLAHTF

SEQ ID NO: 17 - protein, 211 aa, Artificial Sequence
  M 159T
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAETEMGHRYLSVIMQFFGVPSFEGLFVEGHAAVPEKAEEIKANAIARAKDLAHTF

SEQ ID NO: 18 - protein, 211 aa, Artificial Sequence
  A187E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVEGHEAVPEKAEEIKANAIARAKDLAHTF

SEQ ID NO: 19 - protein, 211 aa, Artificial Sequence
  A 187T
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVEGHTAVPEKAEEIKANAIARAKDLAHTF

SEQ ID NO: 20 - protein, 211 aa, Artificial Sequence
  A187V
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVEGHVAVPEKAEEIKANAIARAKDLAHTF

SEQ ID NO: 21 - protein, 211 aa, Artificial Sequence
  R64H/A146T
MTNVLYITAHPHDDTQSYSMAVGKAFIDTYKQVHPDHEVIHLDLYKEYIPEIDVDVFSGWGKLHSGKSFEELSDEEKAKVGRMNELCEQFISADKYVFVTPMWNFSFPPVLKAYIDAVAVAGKTFKYTEQGPVGLLTDKKALHIQTRGGFYSEGPAAEMEMGHRYLSVIMQFFGVPSFEGLFVEGHAAVPEKAEEIKANAIARAKDLAHTF

SEQ ID NO: 22 - protein, 211 aa, Artificial Sequence
  E85D/R147H
MTNVLYITAHPHDDTQSYSMAVGKAFIDTYKQVHPDHEVIHLDLYKEYIPEIDVDVFSGWGKLRSGKSFEELSDEEKAKVGRMNDLCEQFISADKYVFVTPMWNFSFPPVLKAYIDAVAVAGKTFKYTEQGPVGLLTDKKALHIQAHGGFYSEGPAAEMEMGHRYLSVIMQFFGVPSFEGLFVEGHAAVPEKAEEIKANAIARAKDLAHTF

SEQ ID NO: 23 - protein, 211 aa, Artificial Sequence
  F105L/A187E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNLSFPPVLKAYIDAVAVAGKTFKYTEQGPVGLLTDKKALHIQARGGFYSEGPAAEMEMGHRYLSVIMQFFGVPSFEGLFVEGHEAVPEKAEEIKANAIARAKDLAHTF

SEQ ID NO: 24 - protein, 211 aa, Artificial Sequence
  A113E/K126N
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKEYIDAVAVAGKTFNYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 25 - protein, 211 aa, Artificial Sequence
  Y151H/A187E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFHSEGPAAEMEMGHRYLSVIMQFFGVPSFEGLFVEGHEAVPEKAEEIKANAIARAKDLAHTF

SEQ ID NO: 26 - protein, 211 aa, Artificial Sequence
  G122D/A187E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVADKTFKYTEQGPVGLLTDKKALHIQARGGFYSEGPAAEMEMGHRYLSVIMQFFGVPSFEGLFVEGHEAVPEKAEEIKANAIARAKDLAHTF

SEQ ID NO: 27 - protein, 211 aa, Artificial Sequence
  G149D/A1 87E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGDFYSEGPAAEMEMGHRYLSVIMQFFGVPSFEGLFVEGHEAVPEKAEEIKANAIARAKDLAHTF

SEQ ID NO: 28 - protein, 211 aa, Artificial Sequence
  G149S/A187E/L207W
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGSFYSEGPAAEMEMGHRYLSVIMQFFGVPSFEGLFVEGHEAVPEKAEEIKANAIARAKDWAHTF

SEQ ID NO: 29 - protein, 211 aa, Artificial Sequence
  F105L/A187E/L207W
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNLSFPPVLKAYIDAVAVAGKTFKYTEQGPVGLLTDKKALHIQARGGFYSEGPAAEMEMGHRYLSVIMQFFGVPSFEGLFVEGHEAVPEKAEEIKANAIARAKDWAHTF

SEQ ID NO: 30 - protein, 211 aa, Artificial Sequence
  G66R/F105L/A187E/K192R
MTNVLYITAHPHDDTQSYSMAVGKAFIDTYKQVHPDHEVIHLDLYKEYIPEIDVDVFSGWGKLRSRKSFEELSDEEKAKVGRMNELCEQFISADKYVFVTPMWNLSFPPVLKAYIDAVAVAGKTFKYTEQGPVGLLTDKKALHIQARGGFYSEGPAAEMEMGHRYLSVIMQFFGVPSFEGLFVEGHEAVPERAEEIKANAIARAKDLAHTF

SEQ ID NO: 31 - protein, 211 aa, Artificial Sequence
  A146G/L207W
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQGRGGFYSEGPAAEMEMGHRYLSVIMQFFGVPSFEGLFVEGHAAVPEKAEEIKANAIARAKDWAHTF

SEQ ID NO: 32 - protein, 211 aa, Artificial Sequence
  F105L/A187E/Q171P
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNLSFPPVLKAYIDAVAVAGKTFKYTEQGPVGLLTDKKALHIQARGGFYSEGPAAEMEMGHRYLSVIMPFFGVPSFEGLFVEGHEAVPEKAEEIKANAIARAKDLAHTF

SEQ ID NO: 33 - protein, 211 aa, Artificial Sequence
  A78E/F105L/A187E
MTNVLYITAHPHDDTQSYSMAVGKAFIDTYKQVHPDHEVIHLDLYKEYIPEIDVDVFSGWGKLRSGKSFEELSDEEKEKVGRMNELCEQFISADKYVFVTPMWNLSFPPVLKAYIDAVAVAGKTFKYTEQGPVGLLTDKKALHIQARGGFYSEGPAAEMEMGHRYLSVIMQFFGVPSFEGLFVEGHEAVPEKAEEIKANAIARAKDLAHTF

SEQ ID NO: 34 - protein, 211 aa, Artificial Sequence
  F105L/K139N/V168L/A187E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNLSFPPVLKAYIDAVAVAGKTFKYTEQGPVGLLTDNKALHIQARGGFYSEGPAAEMEMGHRYLSLIMQFFGVPSFEGLFVEGHEAVPEKAEEIKANAIARAKDLAHTF

SEQ ID NO: 35 - protein, 211 aa, Artificial Sequence
  G154D/G18OR
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEDPAAEMEMGHRYLSVIMQFFGVPSFERLFVEGHAAVPEKAEEIKANAIARAKDLAHTF

SEQ ID NO: 36 - protein, 211 aa, Artificial Sequence
  F1071
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSIPPVLKAYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF

SEQ ID NO: 37 - protein, 211 aa, Artificial Sequence
  G 185R
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVERHAAVPEKAEEIKANAIARAKDLAHTF

SEQ ID NO: 38 - protein, 211 aa, Artificial Sequence
  Y151H/G185R
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFHSEGPAAEMEMGHRYLSVIMQFFGVPSFEGLFVERHAAVPEKAEEIKANAIARAKDLAHTF

SEQ ID NO: 39 - protein, 211 aa, Artificial Sequence
  G122D/G185R
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVADKTFKYTEQGPVGLLTDKKALHIQARGGFYSEGPAAEMEMGHRYLSVIMQFFGVPSFEGLFVERHAAVPEKAEEIKANAIARAKDLAHTF

SEQ ID NO: 40 - protein, 211 aa, Artificial Sequence
  G149D/G185R
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGDFYSEGPAAEMEMGHRYLSVIMQFFGVPSFEGLFVERHAAVPEKAEEIKANAIARAKDLAHTF

SEQ ID NO: 41 - protein, 211 aa, Artificial Sequence
  G149D/G185R/A208V
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGDFYSEGPAAEMEMGHRYLSVIMQFFGVPSFEGLFVERHAAVPEKAEEIKANAIARAKDLVHTF

SEQ ID NO: 42 - protein, 211 aa, Artificial Sequence
  F107I/G185R
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSIPPVLKAYIDAVAVAGKTFKYTEQGPVGLLTDKKALHIQARGGFYSEGPAAEMEMGHRYLSVIMQFFGVPSFEGLFVERHAAVPEKAEEIKANAIARAKDLAHTF

SEQ ID NO: 43 - protein, 211 aa, Artificial Sequence
  F1071/G185R/A208V
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSIPPVLKAYIDAVAVAGKTFKYTEQGPVGLLTDKKALHIQARGGFYSEGPAAEMEMGHRYLSVIMQFFGVPSFEGLFVERHAAVPEKAEEIKANAIARAKDLVHTF

SEQ ID NO: 44 - protein, 211 aa, Artificial Sequence
  F107I/G185R/Q171P
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSIPPVLKAYIDAVAVAGKTFKYTEQGPVGLLTDKKALHIQARGGFYSEGPAAEMEMGHRYLSVIMPFFGVPSFEGLFVERHAAVPEKAEEIKANAIARAKDLAHTF

SEQ ID NO: 45 - protein, 211 aa, Artificial Sequence
  V80D/F1071/G185R
MTNVLYITAHPHDDTQSYSMAVGKAFIDTYKQVHPDHEVIHLDLYKEYIPEIDVDVFSGWGKLRSGKSFEELSDEEKAKDGRMNELCEQFISADKYVFVTPMWNFSIPPVLKAYIDAVAVAGKTFKYTEQGPVGLLTDKKALHIQARGGFYSEGPAAEMEMGHRYLSVIMQFFGVPSFEGLFVERHAAVPEKAEEIKANAIARAKDLAHTF

SEQ ID NO: 46 - protein, 211 aa, Artificial Sequence
  F1071/K139N/V168L/G185R
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSIPPVLKAYIDAVAVAGKTFKYTEQGPVGLLTDNKALHIQARGGFYSEGPAAEMEMGHRYLSLIMQFFGVPSFEGLFVERHAAVPEKAEEIKANAIARAKDLAHTF

SEQ ID NO: 47 - protein, 211 aa, Artificial Sequence
  F150V
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGVYSEGPAAEMEMGHRYLSVIMQFFGVPSFEGLFVEGHAAVPEKAEEIKANAIARAKDLAHTF

SEQ ID NO: 48 - protein, 211 aa, Artificial Sequence
  A193E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFYSEGPAAEMEMGHRYLSVIMQFFGVPSFEGLFVEGHAAVPEKEEEIKANAIARAKDLAHTF

SEQ ID NO: 49 - protein, 211 aa, Artificial Sequence
  F150V/A193E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGVYSEGPAAEMEMGHRYLSVIMQFFGVPSFEGLFVEGHAAVPEKEEEIKANAIARAKDLAHTF

SEQ ID NO: 50 - protein, 211 aa, Artificial Sequence
  Y151H/A193E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGFHSEGPAAEMEMGHRYLSVIMQFFGVPSFEGLFVEGHAAVPEKEEEIKANAIARAKDLAHTF

SEQ ID NO: 51 - protein, 211 aa, Artificial Sequence
  G122D/A193E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVADKTFKYTEQGPVGLLTDKKALHIQARGGFYSEGPAAEMEMGHRYLSVIMQFFGVPSFEGLFVEGHAAVPEKEEEIKANAIARAKDLAHTF

SEQ ID NO: 52 - protein, 211 aa, Artificial Sequence
  G149D/A193E/A208V
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGDFYSEGPAAEMEMGHRYLSVIMQFFGVPSFEGLFVEGHAAVPEKEEEIKANAIARAKDLVHTF

SEQ ID NO: 53 - protein, 211 aa, Artificial Sequence
  F150V/A193E/A208V
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGVYSEGPAAEMEMGHRYLSVIMQFFGVPSFEGLFVEGHAAVPEKEEEIKANAIARAKDLVHTF

SEQ ID NO: 54 - protein, 211 aa, Artificial Sequence
  F150V/A193E/Q171P
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDKKALHIQARGGVYSEGPAAEMEMGHRYLSVIMPFFGVPSFEGLFVEGHAAVPEKEEEIKANAIARAKDLAHTF

SEQ ID NO: 55 - protein, 211 aa, Artificial Sequence
  V80D/F150V/A193E
MTNVLYITAHPHDDTQSYSMAVGKAFIDTYKQVHPDHEVIHLDLYKEYIPEIDVDVFSGWGKLRSGKSFEELSDEEKAKDGRMNELCEQFISADKYVFVTPMWNFSFPPVLKAYIDAVAVAGKTFKYTEQGPVGLLTDKKALHIQARGGVYSEGPAAEMEMGHRYLSVIMQFFGVPSFEGLFVEGHAAVPEKEEEIKANAIARAKDLAHTF

SEQ ID NO: 56 - protein, 211 aa, Artificial Sequence
  K139N/F150VN168L/A193E
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNFSFPPVLKAYIDAVAVAGKTFKYTEQGPVGLLTDNKALHIQARGGVYSEGPAAEMEMGHRYLSLIMQFFGVPSFEGLFVEGHAAVPEKEEEIKANAIARAKDLAHTF

SEQ ID NO: 57 - DNA, 33 bp, Artificial Sequence
  Primer 1
ggaattccatatgatgacaaacgtattgtacat

SEQ ID NO: 58 - DNA, 28 bp, Artificial Sequence
  Primer 2
cgggatccttaaaacgtgtgcgccaagt

SEQ ID NO: 59 - DNA, 33 bp, Artificial Sequence; identical to SEQ ID NO: 57
  Primer 3

SEQ ID NO: 60 - DNA, 28 bp, Artificial Sequence
  Primer 4
cgggatccttaaaacgtgtgcgccaagt